(19) United States
(12) Patent Application Publication
(10) Pub. No.: US 2008/0248572 A1
(43) Pub. Date: Oct. 9, 2008

(54) Bioreactor Surfaces

(75) Inventor: Glen Delbert ANTWILER, Lakewood, CO (US)

Correspondence Address: GAMBRO BCT, INC;IP DEPARTMENT, 10810 WEST COLLINS AVE, LAKEWOOD, CO, 80215, US

(73) Assignee: GAMBRO BCT, INC., Lakewood, CO (US)

(21) Appl. No.: 12/042,541

(22) Filed: Mar. 5, 2008

Related U.S. Application Data
(60) Provisional application No. 60/910,502, filed on Apr. 6, 2007.

(51) Int. Cl. C12N 5/00; C12M 3/00

(52) U.S. Cl. 435/400; 435/289.1

(57) ABSTRACT

The present invention relates to treatment of polymeric bioreactor surfaces, to promote the proliferation of adherent cells.

### CROSS-REFERENCE TO RELATED APPLICATION

This application claims the benefit of U.S. Provisional Patent Application No. 60/910502, filed Apr. 6, 2007.

### BACKGROUND OF THE INVENTION

Stem cells are a category of undifferentiated cells that demonstrate potential in various therapeutic applications, including organ transplantation, tissue regeneration, blood transfusion, and bone marrow transplantation. To grow stem cells in amounts useful for therapeutic applications, an efficient and reliable mechanism for expanding stem cells is important. To be economically useful, such a mechanism should ensure that large numbers of stem cells are produced, in a manner that minimizes the chances of contaminating the stem cells.

Mammalian cells require homeostasis to survive; therefore, when growing human cells ex vivo, certain environmental parameters, including temperature, oxygen concentration, pH, osmolarity, nutrient concentrations, and ion concentrations must be carefully regulated. Further, in the context of stem cell expansion, many frequently grown cells such as mesenchymal stem cells (MSC), are anchorage-dependent. This means that when expanding MSCs ex vivo, their viability and proliferative capacity may be diminished unless they become anchored to a fixed surface.

There thus arises a need for a system in which cells can be grown efficiently in immobilized culture while minimizing labor costs and contamination risks. The most rudimentary bioreactor system involves the use of a polystyrene tissue-culture flask, but this method is impractical for all but the smallest-scale applications: culturing cells in culture flasks is labor-intensive, and has a high risk of contamination, owing to the fact that frequent opening of a culture flask increases the probability of contamination in the flask. Many bioreactor systems developed in response to the problems associated with tissue-culture flasks are referred to as “closed” systems; in these systems, automated fluid flow delivers nutrients to the cells, therefore allowing fewer chances for contamination, affording improved control over the process, and better approximating the cells' physiological environment. Automated culture bioreactors may be of the flat-plate or the hollow-fiber variety; use of the hollow-fiber variety maximizes the surface area available for growing cells within a reactor of a given volume.

Consequently, effort has been devoted to developing bioreactor surfaces to which adherent cells will attach. The task of developing suitable surfaces for cell adhesion has been made more difficult by the fact that cells do not readily or tightly adhere to the materials from which present-day hollow fibers are made. A solution to these problems requires the development of a cell adhesion system capable of adhering cells to surfaces to which they do not naturally bind, and to keep the cells bound in spite of constant exposure to shear stresses caused by the flow of media over the cells.

The polymers used as the substrata in constructing cell growth surfaces in known membrane bioreactors include polystyrene, polypropylene, polyethylene, polymethylpentylene, saponified cellulose esters, polymethylacrylate, polycarbonate, polyesters, polyethersulfone, styrene-acrylonitrile, polyacrylonitrile, PVC, organosilicone, cellulose ester, and polyamide.

Some polymeric surfaces have been treated with cell adhesion factors such as laminin, collagen, and fibronectin, either via covalent attachment or electrostatic adsorption. Other surfaces are treated using cell adhesion factors to physically anchor cells to membrane surfaces as shown in U.S. Pat. No. 5,912,177. However, the use of cell adhesion factors by themselves is recognized in the art as insufficient to mediate the long-term attachment of cells to a polymeric matrix, as described in U.S. Pat. Nos. 5,512,474; 5,912,177, and as described, for example, by Prichard, H., Reichert, W. et al. in “Adult Adipose-Derived Stem Cell Attachment to Biomaterials” Biomaterials 28(6) 936-946. Furthermore, fibronectin and other cell-adhesion factors are expensive, and the current adsorptive techniques for fibronectin coating of polymeric surfaces are highly wasteful.

It is therefore desirable to find ways of either using fibronectin more efficiently, or to find ways of obviating the need for fibronectin.

### BRIEF DESCRIPTION OF THE FIGURES

FIG. 1 is a schematic view of a bioreactor useful in this invention.

FIG. 2 is a flow diagram of a cell expansion system that may be used with the present invention.

FIG. 3 is a graph comparing the effects of various surface treatments on mesenchymal stem cell growth.

FIG. 4 is another graph comparing the effects of various surface treatments on mesenchymal stem cell growth.

### SUMMARY OF THE INVENTION

This invention is directed towards a method for promoting adhesion of mammalian cells to a membrane surface in a bioreactor. The bioreactor has at least a housing and a polymeric membrane having at least one surface inside the housing. The polymeric membrane surface is treated with at least one surface treatment in an amount sufficient to improve cell adhesion to the polymeric membrane surface.

Another embodiment includes the improved cell culture system; the cell culture system comprises a bioreactor comprising a housing and polymeric membrane inside the housing; and in which the polymeric membrane is treated with platelet lysate or with plasma, in an amount sufficient to stimulate cell binding and adhesion to the membrane.

Another embodiment of the invention includes a cell culture surface for use in a cell culture system. This cell culture surface is a polymeric material treated with platelet lysate or plasma or combinations thereof in an amount sufficient to promote cell adhesion to the polymeric material.

### DETAILED DESCRIPTION OF THE INVENTION

As discussed in the background, there are multiple ways of configuring a bioreactor in order to grow adherent mammalian cells; this invention is not dependent on any one configuration thereof. As used herein, the term “adherent mammalian cells” refers to any type of eukaryotic cells possessing a mammalian nuclear genome and adherent potential, regardless of species of origin, tissue of origin, cell lineage, or length of time in culture.

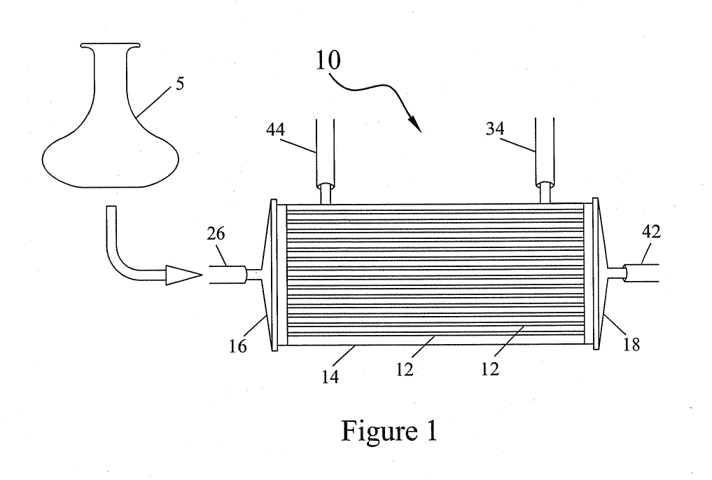

One non-limiting example of the embodiments of the present invention is the hollow-fiber bioreactor shown in FIG. 1. The bioreactor, or cell-expansion module 10 is made from a bundled set of biocompatible polymeric membranes 10 in the geometric form of hollow fibers, enclosed within a housing 14. For purposes of this description, the set of all the hollow fibers, and both the intracapillary (IC) and extracapillary (EC) sides of them, is referred to as a membrane. The terms “membrane”, “cell culture surface”, “culture surface”, “polymeric membrane”, and “polymeric surface” are synonymous. The housing, or module 14 containing the fibers 12 may be cylindrical in shape, and may be made from any biocompatible polymeric material. The intracapillary side of the membrane is defined for purposes of this description as the luminal side of, and the volume enclosed by, or substantially enclosed by, a membrane resembling a hollow fiber. The extracapillary side of the membrane is defined for purposes of this description as any component of the volume within the bioreactor housing that is not enclosed by, or in contact with the luminal side of the hollow fibers. For purposes of this description, it is assumed that cells will be seeded, grown, and reseeded in the IC space only; this assumption is not intended to limit the scope of the claims, and it is understood that in the alternative, cells could be grown in the EC space, and the same principles described here would apply.

Each end of the module, or housing, is closed off with end caps, or headers 16, 18. These end caps 16, 18 may be made of any suitable material such as polycarbonate so long as the material is biocompatible with the types of cells to be grown in the bioreactor.

The module has at least one port for entry and exit of fluids into the module; the module of an embodiment, as a nonlimiting example, has four ports. Two of the four ports fluidly connect to the extracapillary space. One port 34 is used for fluid and solute ingress into the extracapillary space, and the other port 44 is used for fluid and solute egress from the extracapillary space. The other two of the four ports fluidly connect to the intracapillary space; as a nonlimiting example, one port 26 is used for fluid and solute ingress into the intracapillary space, and the other port 42 is used for fluid and solute egress from the intracapillary space. It is by means of the aforesaid inlet ports 34, 26 that cells, treatments, and media may be introduced into the bioreactor, and it is by means of the egress ports 44, 42 that cells, treatments, and media may be removed from the bioreactor at times. For purposes of the invention, any physical aperture in a bioreactor 10 that allows ingress of material into the bioreactor is an inlet port; any port through which egress of material from the bioreactor occurs is an egress port.

The IC space is assumed to serve as a cell-growth chamber; however, as stated before, this assumption is nonessential to the invention, as cells may also be flowed into, and grown in the EC space. At the start of a new cell expansion period, cells may be flowed into the IC space. The IC space may be loaded with cells using a syringe, or from a bag containing a preparation of cells. The cells may be flowed into the IC space in cell culture media, or directly as bone marrow aspirate.

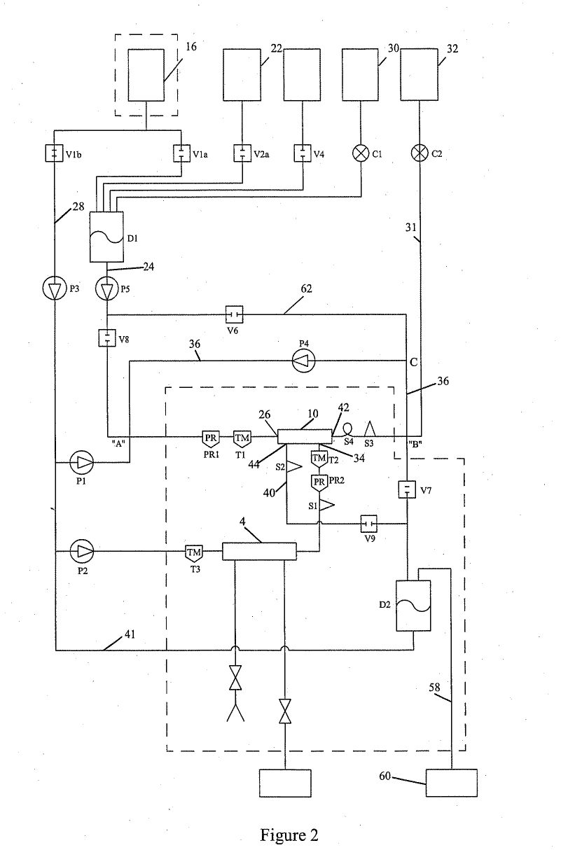

In an embodiment, an IC media bag 22 (see FIG. 2) may be connected via a portion of flexible tubing (the IC inlet line) 24 to the IC inlet port 26 of the bioreactor 10. The IC inlet line 24 brings fresh IC media to the IC side of the bioreactor. Additional tubing line 62 can be added to the system as needed to enable specific applications such as reseeding or redistributing cells in the bioreactor.

A cell input bag 30 contains the cells to be expanded in the bioreactor 10. The cell input bag 30 is connected to the IC inlet line 24 that delivers cells into the lumen of the hollow fibers via IC inlet port 26.

When the cells are ready to be harvested, they are flushed out of the IC outlet port 42 of bioreactor 10 through cell harvest line 31 and into a cell harvest bag 32.

The cell growth system also may include a length of tubing which acts as an IC re-circulation loop 36. The IC media flows out of the bioreactor 10 from the IC outlet port 42 through tubing loop 36 and back into the bioreactor through the IC inlet port 26. This loop 36 is used to recirculate the IC media though the hollow fibers. It may also be used to flush the cells out of the hollow fibers and reseed/redistribute them throughout the hollow fibers for further expansion as more fully described below.

As seen in FIG. 1, the space between the fibers 12 themselves, or EC space, may serve as a nutrient reservoir and a waste-collection site for the cells in the intracapillary space. Nutrients enter the IC space from the EC space by means of diffusion across the polymeric membrane; further, cellular waste products leave the IC space via the EC space. The EC media may be replaced at intervals to remove cell metabolic wastes, or may be continuously replaced. The EC media may be circulated as needed through an oxygenator (4, see FIG. 2). The EC media may be introduced into the bioreactor from an EC media bag (16, see FIG. 2), which in an embodiment is fluidly connected via a length of flexible tubing, or conduit 28 to EC inlet port 34. The EC media, along with any cellular wastes, may be flushed from the bioreactor via EC egress port 44, which is fluidly connected through a length of flexible tubing, or conduit 58, to a waste bag 60.

Also an EC recirculation loop including lines 40 and 41 may be provided to recirculate EC media. Again, if cells were being grown in the EC space, the IC media would serve as a nutrient reservoir and a waste collection pool for the cells.

A second assumption made solely for purposes of this description is that the fluid flowing through the IC space and the fluid flowing through the EC space flow opposite directions. This assumption is nonessential to the invention, as the invention may be used in a bioreactor in which fluid flows the same direction in both the EC and IC spaces.

The hollow fibers 12 in the particular embodiments here described are approximately 9000 in number, and are approximately 295 mm in length. They may be held in place within the housing by polyurethane potting (not shown). The fibers 12 and the potting may be cut through cross-sectionally, to permit fluid flow through the IC space. It is understood that the length and number of the fibers 12 may be varied; the embodiments here described are merely exemplary.

The hollow fibers 12 may be made of a semi-permeable, biocompatible, polymeric material. One such polymeric material is a blend of polyamide and polyarylethersulfone. The semi-permeable membrane allows transfer of nutrients, wastes, and gases through the membrane between the EC and IC spaces. Exchange of fluid takes place in part because the fibers have a generally porous consistency, which facilitates diffusion and convection of molecules across the membranes.

One embodiment of the membrane 12 comprises 65-95% by weight of at least one hydrophobic polymer and 5-35% by weight of at least one hydrophilic polymer. The hydrophobic polymer may be chosen from the group consisting of polyamide (PA), polyaramide (PAA), polyarylethersulfone (PAES), polyethersulfone (PES), polysulfone (PSU), polyarylsulfone (PASU), polycarbonate (PC), polyether (PE), polyurethane (PUR), polyetherimide, and copolymer mixtures of any of the above polymers, such as polyethersulfone, or a mix of polyethersulfone and polyamide. The hydrophilic polymer may be chosen from the group consisting of polyvinylpyrrolidone (PVP), polyethylene glycol, (PEG), polyglycolmonoester, water-soluble cellulosic derivatives, polysorbate, and polyethylene-polypropylene oxide copolymers.

The polymeric hollow fibers 12 may be treated with a substance, or “surface treatment” to improve the adherence of the cells to the membrane, especially if adherent cells, or anchorage-dependent cells are to be grown in the bioreactor. The terms “treat”, “treated”, or “treating” mean that substantially all portions of the cell culture surfaces of the hollow fibers have been subjected to a surface treatment for an amount of time sufficient to allow the treatment molecules to become adsorbed to the membrane. Further, covalent, adsorbed, and soluble treatments may be used in conjunction with one another, without restriction as to combinations or amounts.

### Methods

The steps of treating the membrane or cell culture surface with a surface preparation may be conducted as follows: prior to the membrane treating step, the cell culture surface 12 is primed by wetting with a saline solution, which in an embodiment is PBS, or phosphate buffered saline. To avoid the formation of precipitates, the PBS must be free of divalent cations such as Mg⁺⁺ or Ca⁺⁺.

Following the priming procedure, the membrane is treated with a surface treatment, such as platelet lysate (PL), plasma, and fibronectin (FN).

For purposes of this invention, human platelet lysate is a solution containing plasma and lysed human platelets. The solution may be prepared by any method of causing human platelets to lyse, including those methods currently known in the art. In one method, 1.5×10⁹/mL platelets in plasma is frozen at −80° C. to lyse the platelets; the resulting biologically active solution is hereafter known as platelet lysate solution. This platelet lysate is thawed and centrifuged at 1000×g for 10 minutes. The resulting supernatant is used to treat the membrane.

For purposes of this invention, plasma consists of any preparation of human plasma from which substantially all leukocytes and erythrocytes have been removed, by any method, including those known in the art. The platelet content of the plasma may vary.

For purposes of this invention, the surface treatment may also be fibronectin (FN), dissolved in PBS at a concentration of 0.05 mg/mL.

The surface treatment is allowed to contact the membrane 12 by pumping or dripping the surface treatment into the IC space.

The surface treatment may be introduced into the bioreactor by itself, or may be included in the cell culture media. In an alternate embodiment, one surface treatment may be introduced into the bioreactor along with another surface treatment which is different from the first surface treatment.

Once in contact with the membrane of the bioreactor, the surface treatment is allowed to incubate with the membrane for an amount of time sufficient to allow adsorption of the surface treatment in an amount sufficient to promote enhanced cell adhesion. In one embodiment of the invention, a fibronectin treatment solution is allowed to incubate with the cell culture surface for at least one hour.

Cell loading may be accomplished by sending aqueously-suspended cell samples into the bioreactor 10 via the IC inlet port 26. As discussed above, platelet lysate or plasma may also be included with the cells to be expanded.

### EXAMPLES

### Example 1

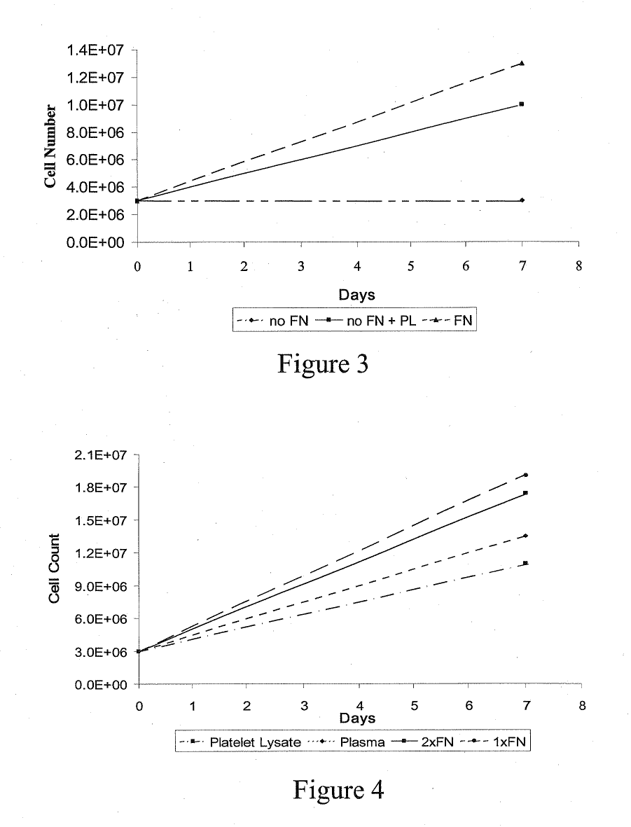

Three polyflux hollow fiber bioreactors were used in this example. One bioreactor-was not treated with anything (referred to in FIG. 3 as no FN). One bioreactor was treated with fibronectin (FN) and one was treated with platelet lysate (no FN+PL) according to the above-described methods. Around 3×10⁶ mesenchymal stem cells were loaded into each bioreactor on day 0. The cells were grown for seven days. The EC and IC media was replaced on days three and five and the cells were harvested and counted on day seven.

As can be seen from FIG. 3, the bioreactors treated with either fibronectin (FN) or platelet lysate (PL) produced much better cell expansion than the untreated bioreactor. Increased cell numbers produced by the bioreactors with the treated fibers indicate that cells were able to attach to the membrane and grow.

### Example 2

Four polyflux hollow fiber bioreactors were used in this example. One bioreactor was treated with an amount of fibronectin (1× FN), one bioreactor was treated with twice the amount of fibronectin (2× FN), one bioreactor was treated with platelet lysate and one was treated with plasma according to the above-described methods. Around 3×10⁶ mesenchymal stem cells were loaded into each bioreactor on day 0. The cells were grown for seven days. The EC and IC media was replaced on days three and five and the cells were harvested and counted on day seven.

As can be seen from FIG. 4, the bioreactors treated with either 1× or 2× fibronectin produced the highest cell expansion. However, cells grown on membranes treated with platelet lysate and plasma also showed good expansion in culture.

The examples given above are several of the applications which could be utilized following the principals of the present invention and are not meant to limit the spirit and scope of the present invention as defined by the attached claims.

1-5. (canceled)

6. The method of claim 12 wherein the step of treating the intracapillary space surface of the polymeric membrane with at least one surface treatment further comprises treating the membrane surface with a second surface treatment which is different from the first surface treatment.

7. A cell culture system for ex vivo culturing cells, comprising;
a bioreactor comprising
a housing and
a polymeric membrane inside the housing;
wherein the polymeric membrane is treated with platelet lysate in an amount sufficient to improve cell adherence to the membrane.

8. The system of claim 7, wherein the polymeric membrane is a hollow fiber membrane.

9. The system of claim 7, wherein the polymeric material comprises at least one hydrophobic polymer and at least one hydrophilic polymer.

10. A surface for use in a cell culture system comprising a polymeric material treated with platelet lysate.

11. A surface for use in a cell culture system comprising a polymeric material treated with plasma.

12. A method for promoting adhesion of mammalian cells to a membrane surface in a bioreactor comprising:
providing a bioreactor comprising
a housing; and
a hollow fiber semi-permeable polymeric membrane having an intracapillary space surface and an extracapillary space surface
wherein the polymeric membrane comprises at least one hydrophobic polymer and at least one hydrophilic polymer; and
treating the intracapillary space surface of the polymeric membrane with at least a first surface treatment selected from the group consisting of platelet lysate or plasma in an amount sufficient to improve cell adhesion to the intracapillary space membrane surface.
